(19) United States
(12) Patent Application Publication
(10) Pub. No.: US 2014/0277431 A1
(43) Pub. Date: Sep. 18, 2014

(54) TREATMENTS OF EXTRACELLULAR MATRICES OF THE EYE

(71) Applicant: AVEDRO, INC., Waltham, MA (US)

(72) Inventors: Satish Herekar, Palo Alto, CA (US); David Muller, Boston, MA (US); Marc D. Friedman, Needham, MA (US)

(73) Assignee: AVEDRO, INC., Waltham, MA (US)

(21) Appl. No.: 14/216,291

(22) Filed: Mar. 17, 2014

Related U.S. Application Data
(60) Provisional application No. 61/792,463, filed on Mar. 15, 2013.

(51) Int. Cl. A61F 2/16

(52) U.S. Cl. CPC A61F 2/16
USPC 623/6.12

(57) ABSTRACT

System and methods for a corrective eye procedure include at least one application device configured to be positioned at a selected area of an eye (e.g., equatorial sclera, posterior sclera, cornea, etc.). The at least one device includes at least one channel and at least one illumination guide. A cross-linking agent source is coupled to the at least one channel. An illumination source is coupled to the at least one illumination guide. The at least one device delivers the cross-linking agent to the selected area of the eye. The at least one device delivers photo-activating light from the illumination source to the selected area of the eye after the cross-linking agent has been delivered. The photo-activating light includes one or more doses necessary for activating the cross-linking agent and for activating TGF-β isoforms to improve health of extracellular matrices in the selected area of the eye.

### CROSS REFERENCE TO RELATED APPLICATIONS

This application claims priority to U.S. Provisional Patent Application No. 61/792,463, filed Mar. 15, 2013, the contents of which are incorporated entirely herein by reference.

### FIELD OF THE INVENTION

The present invention relates generally to systems and methods for treating eye disorders, and more particularly, to systems and methods that treat extracellular matrices of the eye to address disorders, such as scleral progressive myopia.

### BACKGROUND

Pathological extracellular matrices (ECM) of the eye are implicated in keratoconus (KCN) and scleral progressive myopia due to tissue structural instabilities. For example, these disorders are prevalent in 0.05% and 30% of Asian populations, respectively, while being prevalent in 0.05% and 2% of U.S./European populations, respectively. Cross-linking of corneal tissue provides treatment for KCN, but treatments of the posterior and equatorial sclera to treat scleral progressive myopia are far more invasive to implement, often requiring 360 degree peritomies and rectus muscle/Tenon's manipulation in young patients when implanting scleral buckles, for example.

Equatorial and posterior scleral (fibrillar) thinning are the initial signs of scleral progressive myopia due mainly to a loss of collagen tissue resulting from biochemical imbalances/pathologies (such as inhibition of lysyl oxidsase activity). Studies report 35% reduction in collagen type I mRNA indicating collagen production is decreased at the same time that ECM degradation increases. Similarly, glycosaminoglycans (GAGs, hence proteoglycans) have been shown to be diminished with a net negative change in the ECM, although DNA synthesis appears unaltered. Bio-mechanical thinning is accompanied by significantly increased scleral creep (>200%). Altered integrin expression, and reduced fibroblast to myofibroblast differentiation are also noted. In all, the confluence of these conditions results in scleral elongation under physiologic intraocular pressure (IOP) but with reduced collagen content (˜7%).

### SUMMARY

Aspects of the present invention provide systems and methods that improve the health of the extracellular matrices (ECM) by modulation of transforming growth factor beta (TGF-β) isoforms, which are cytokines known to be involved in cell growth inhibition, embryogenesis, differentiation, wound healing and apoptosis in part. Aspects of the present invention remodel scleral and/or corneal ECM via growth factor activation in combination with additional treatments such as cross-linking and exogenous cytokine augmented repair of ECM that are the primary determinant of follow-on high myopia and/or corneal ectasia.

According to one example embodiment, a system for conducting a corrective scleral procedure for an eye, includes at least one insert configured to be positioned at a selected area of scleral tissue (e.g., equatorial sclera, posterior sclera, etc.). The at least one insert includes at least one channel and at least one illumination guide. A cross-linking agent source is coupled to the at least one channel. An illumination source is coupled to the at least one illumination guide. The at least one insert delivers the cross-linking agent to the selected area of scleral tissue via the at least one channel. The at least one insert delivers photo-activating light from the illumination source to the selected area of scleral tissue via the at least one illumination guide after the cross-linking agent has been delivered. The photo-activating light includes one or more doses necessary for generating cross-linking activity in the scleral tissue by activating the cross-linking agent and for activating TGF-β isoforms for improving health of extracellular matrices in the selected area of scleral tissue.

According to another example embodiment, a method for a corrective scleral procedure includes positioning at least one insert at a selected area of scleral tissue. The at least one insert includes at least one channel and at least one illumination guide. A cross-linking agent source is coupled to the at least one channel. An illumination source being coupled to the at least one illumination guide. The method also includes delivering the cross-linking agent to the selected area of scleral tissue via the at least one channel. The method additionally includes delivering photo-activating light from the illumination source to the selected area of scleral tissue after the cross-linking agent has been delivered. The photo-activating light includes one or more doses necessary for generating cross-linking activity in the scleral tissue by activating the cross-linking agent and for activating TGF-β isoforms to improve health of extracellular matrices in the selected area of scleral tissue.

According to yet another example embodiment, a system for conducting a corrective procedure for an eye includes a contact lens structure, which includes at least one channel and at least one illumination fiber. The contact lens is configured for application over at least a cornea of the eye. A cross-linking agent source is coupled to the at least one channel. An illumination source is coupled to the at least one illumination fiber. The contact lens structure delivers the cross-linking agent to a selected area of the eye via the at least one channel. The contact lens structure delivers photo-activating light from the illumination source to the selected area of the eye via the illumination fiber after the cross-linking agent has been delivered. The photo-activating light including one or more doses necessary for generating cross-linking activity in the scleral tissue by activating the cross-linking agent and for activating TGF-β isoforms to improve health of extracellular matrices in the selected area of the eye.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIGS. 1A, B illustrate an example equatorial insert for delivering oxygen, photosensitizers, saline, exogenous materials, vacuum suction, etc., for a scleral procedure, according to aspects of the present invention.

FIGS. 2A, B illustrate example quad inserts for delivering activation illumination as well as oxygen, photosensitizers, saline, exogenous materials, vacuum suction, etc., for a scleral procedure, according to aspects of the present invention.

FIG. 3 illustrates an example contact lens for delivering treatment, according to aspects of the present invention.

FIG. 4 illustrates an example procedure for a corrective scleral procedure, according to aspects of the present invention.

While the invention is susceptible to various modifications and alternative forms, a specific embodiment thereof has been shown by way of example in the drawings and will herein be described in detail. It should be understood, however, that it is not intended to limit the invention to the particular forms disclosed, but on the contrary, the intention is to cover all modifications, equivalents, and alternatives falling within the spirit of the invention.

### DESCRIPTION

Aspects of the present invention provide systems and methods that improve the health of extracellular matrices (ECM) by modulation of transforming growth factor beta (TGF-β) isoforms, which are cytokines known to be involved in cell growth inhibition, embryogenesis, differentiation, wound healing and apoptosis in part. Aspects of the present invention remodel scleral and/or corneal ECM via growth factor activation in combination with additional treatments such as cross-linking and exogenous cytokine augmented repair of the ECM, which are determinants of follow-on high myopia and/or corneal ectasia.

Roles of reactive oxygen species (ROS)-mediated activation of latent TGF-β isoforms in the ECM by photo-bio-modulation (i.e., use of low irradiance of near-infrared (NIR)/visible (VIS) wavelengths at less than approximately 10 J/cm² dosage) have been investigated for dental wound healing applications and result in improved, denser, and better organized collagen formation. Aspects of the present invention significantly enhance these methods for application to the cornea and sclera (equatorial and posterior in particular) through the choice of wavelength and dosage in addition to simultaneous spatial deposition with oxygen. The wavelength and dosage may depend on the thickness of the scleral tissue, as well as safety considerations (e.g., at a wavelength of 365 nm, the dosage may be limited to 32 J/cm²). The results of these enhanced methods disclosed herein are effective, retina-safe, and efficient for cross-linking and exogenous cytokine/growth factor (e.g., epidermal growth factor (EGF))/anti-oxidant (e.g., PRDX6)-mediated augmented repair. In one aspect, the NIR/VIS light is applied to increase activated TGF-β for collagen type I ECM deposition via low doses. In another aspect, NIR/VIS light is applied to activate eosin-mediated collagen cross-linking Cross-linking via application of Riboflavin and photoactivating ultraviolet A (UVA) light can be optionally included with this method. Although cross-linking activity may be a goal, embodiments provide treatments that not only halt the progression of scleral elongation/corneal ectasia but that also normalize the collagen ECM for long term benefits.

FIG. 4 illustrates a procedure 400 employing aspects of the present invention for a corrective scleral procedure, e.g., to address scleral progressive myopia. As shown in FIG. 4, a 360° peritomy is conducted in act 402 (i.e., circumcorneal incision through the conjunctiva). After muscle fixation in act 404, act 406 positions an equatorial insert (one) and oblique quad band inserts (four) along the equatorial sclera and the posterior sclera, respectively. The positioning of the equatorial insert and the quad band inserts in act 406 avoids contact with the ocular muscles. In act 408, the equatorial insert and quad band inserts are used to apply oxygen to the scleral tissue. In act 410, the equatorial insert and quad band inserts are used to apply photosensitizers, e.g., eosin, Riboflavin, etc., to generate cross-linking activity in the scleral tissue (e.g., to strengthen the scleral tissue against scleral elongation). In act 412, the equatorial insert and quad band inserts are used to apply exogenous materials, e.g., collagen, cytokines-EGF, etc., to the scleral tissue for exogenous cytokine augmented repair of the ECM. In act 414, the equatorial insert and quad band inserts are used to apply a saline wash to the scleral tissue. As described further below, the equatorial insert and quad band inserts may include flow channels and ports, micro-fluidic sponges, etc., for receiving and delivering the oxygen, photosensitizers, exogenous materials, and saline in acts 408-414. In act 416, the equatorial insert and oblique quad band inserts illuminate the treated scleral tissue, e.g., with NIR, VIS, and/or UVA light, to activate TGF-β isoforms. This illumination improves the health of the ECM in addition to activating the photosensitizers to generate cross-linking activity. As further described below, the equatorial insert and quad band inserts may include light-guides/introducers for receiving and delivering the illumination of act 416. The conjunctiva is then closed in act 418.

FIGS. 1A, B illustrate an example equatorial insert 100, which may be employed to conduct a corrective scleral procedure, e.g., the procedure 400. The equatorial insert is configured to be applied about the equator of the eye and may have a total thickness of approximately 1 mm. The equatorial insert 100 includes a flow channel 110 for receiving oxygen, photosensitizers, exogenous materials, saline, and vacuum suction, etc., from respective sources 10a-e. External pumps may be employed to deliver these elements to the equatorial inert 100. The equatorial insert 100 also includes a plurality of ports 112 for uniformly delivering these elements from the flow channel 110 to the scleral tissue. In addition, the equatorial insert 100 may include light-guide(s)/introducer(s) 120 to receive and deliver illumination (e.g., NIR, VIS, and/or UVA light) from an illumination source 20 to the scleral tissue.

Correspondingly, FIGS. 2A, B illustrate example oblique quad band inserts 200 that may be used in combination with the equatorial insert 100 of FIGS. 1A, B to conduct a corrective scleral procedure, e.g., the treatment 400. The quad band inserts 200 are configured to be applied to the posterior sclera and may have dimensions of approximately 80 mm×8 mm×0.5 mm. As shown in FIG. 2B, the quad band inserts 200 together sufficiently cover the posterior sclera while avoiding contact with the ocular muscles. In particular, the quad band inserts 200 are configured to accommodate and avoid the recti muscles. As any contact with, or exposure of, the optic nerve should be avoided, a radius of curvature (ROC) of approximately 3 mm is also provided at the distal end of each quad band inserts 200.

The quad band inserts 200 can be applied with an introducer bag or standard retinal instruments (e.g., retractor, etc.). Each quad band insert 200 may also include a visible micro-LED which can be seen from the anterior side by the surgeon to facilitate the proper positioning of the quad band insert 200. The use of a small endoscopic camera may also be employed during application of the quad band inserts 200.

Flow channels 210 are embedded in the quad band inserts 200 for receiving oxygen, photosensitizers, saline, exogenous materials, and vacuum suction, etc., from their respective sources. The elements may be uniformly delivered from the flow channels 210 via micro-fluidic mechanisms 212. For example, the quad band inserts 200 may include micro-fluidic sponges that allow the elements to be delivered through micro-perforations. (In some embodiments, the equatorial insert 100 described above may also employ micro-fluidic mechanisms.)

Like the equatorial insert 100, the quad band inserts 200 may also include light-guide(s)/introducer(s) 220 (disposed along the structure of the quad band inserts 200) to receive and deliver illumination (e.g., NIR, VIS, and/or UVA light) from an illumination source to the scleral tissue. Accordingly, in an example application, the quad band inserts 200 may provide illumination with greater than approximately 80% uniformity and at greater than approximately 50 mW/cm² at a ROC of less than approximately 12 mm.

Aspects of the equatorial inserts 100 and the quad band inserts 200 may be formed from any combination of appropriate flexible materials, available for example from Biomedical Structures (Warwick, R.I.), Secant Medical, Inc. (Perkasie, Pa.), TissueGen, Inc. (Dallas, Tex.). In addition, the equatorial inserts 100 and the quad band inserts 200 may include single face emitting light-guides, available for example from Nanocomp Oy Ltd (Lehmo, Finland). The light-guides may be configured to limit illumination to targeted tissue/structures. In some embodiments, masks or other shielding techniques may be employed to prevent illumination from reaching other more sensitive tissue/structures.

The equatorial insert 100 and the quad band inserts 200 provide an effective system for flushing, soaking, and oxygenating the equatorial and posterior sclera according to a corrective scleral procedure, e.g., to address scleral progressive myopia. The system also provides NIR, VIS, and/or UVA light, to activate TGF-β isoforms in addition to activating cross-linking agents.

Aspects of the present invention are not limited to application to the equatorial and posterior sclera. For example, FIG. 3 illustrates a contact lens structure 300 with a fluidic and suction channel 310 (embedded micro-fluidics) to achieve aspects of the present invention in other regions of the eye. In particular, the contact lens structure 300 provides micro-fluidics through channel 310, which delivers oxygen, photosensitizers, saline, exogenous materials, and vacuum suction from source 10a-e to the eye. In addition, the contact lens structure 300 delivers activating illumination via an illumination fiber 320 coupled to an illumination source. The contact lens structure 300 delivers uniform illumination. However, as shown in FIG. 3, an illumination mask 330 may be employed to deliver the uniform illumination to selected regions of the eye, as for example, in order to induce refractive corneal reshaping.

Aspects of the present invention may employ a monitoring system that may be employed to monitor the systems and methods described herein, e.g., measure the effect of the methods. Additionally, the systems may include a controller to control aspects of the operation of the systems. The controller may be communicatively coupled to the monitoring system to process the images, data, etc., from the monitoring system and to determine any necessary response to such feedback.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

What is claimed is:

1. A system for conducting a corrective scleral procedure for an eye, comprising:
at least one insert configured to be positioned at a selected area of scleral tissue, the at least one insert including at least one channel and at least one illumination guide;
a cross-linking agent source coupled to the at least one channel; and
an illumination source coupled to the at least one illumination guide,
wherein the at least one insert delivers the cross-linking agent to the selected area of scleral tissue via the at least one channel, and
the at least one insert delivers photo-activating light from the illumination source to the selected area of scleral tissue after the cross-linking agent has been delivered, the photo-activating light including one or more doses necessary for generating cross-linking activity in the scleral tissue by activating the cross-linking agent and for activating TGF-β isoforms to improve health of extracellular matrices in the selected area of scleral tissue.

2. The system of claim 1, wherein the at least one insert includes an equatorial insert configured to be positioned about the equator of the eye.

3. The system of claim 1, wherein the at least one insert includes a plurality of band inserts configured to be positioned about the posterior sclera, the band inserts being shaped to prevent contact with ocular muscles or the optic nerve.

4. The system of claim 1, wherein the at least one insert includes: an equatorial insert configured to be positioned about the equator of the eye and a plurality of band inserts configured to be positioned about the posterior sclera, the band inserts being shaped to prevent contact with ocular muscles or the optic nerve.

5. The system of claim 1, further comprising an exogenous material source coupled to the at least one channel, wherein the at least one insert delivers exogenous material to the selected area of scleral tissue, the exogenous material generating exogenous cytokine augmented repair of extracellular matrices.

6. The system of claim 1, further comprising at least one of an oxygen source, a vacuum source, or a saline source coupled to the at least one channel, wherein the at least one insert correspondingly delivers oxygen, vacuum suction, or saline to the selected area of scleral tissue.

7. The system of claim 1, wherein the at least one insert includes a micro-fluidic material, wherein the at least one insert delivers the cross-linking agent to the selected area of scleral tissue according to micro-fluidic mechanisms.

8. The system of claim 1, wherein the illumination source provides at least one of near-infrared (NIR) light, visible (VIS) light, or ultraviolet (UV) light.

9. A method for a corrective scleral procedure, comprising:
positioning at least one insert at a selected area of scleral tissue, the at least one insert including at least one channel and at least one illumination guide, a cross-linking agent source being coupled to the at least one channel, and an illumination source being coupled to the at least one illumination guide;
delivering the cross-linking agent to the selected area of scleral tissue via the at least one channel, and
delivering photo-activating light from the illumination source to the selected area of scleral tissue after the cross-linking agent has been delivered, the photo-activating light including one or more doses necessary for generating cross-linking activity in the scleral tissue by activating the cross-linking agent and for activating TGF-β isoforms to improve health of extracellular matrices in the selected area of scleral tissue.

10. The method of claim 9, wherein the at least one insert includes an equatorial insert configured to be positioned about the equator of the eye.

11. The method of claim 9, wherein the at least one insert includes a plurality of band inserts configured to be positioned about the posterior sclera, the band inserts being shaped to prevent contact with ocular muscles or the optic nerve.

12. The method of claim 9, wherein the at least one insert includes: an equatorial insert configured to be positioned about the equator of the eye and a plurality of band inserts configured to be positioned about the posterior sclera, the band inserts being shaped to prevent contact with ocular muscles or the optic nerve.

13. The method of claim 9, further comprising delivering exogenous material to the selected area of scleral tissue from an exogenous material source, the exogenous material source being coupled to the at least one channel, the exogenous material generating exogenous cytokine augmented repair of extracellular matrices.

14. The method of claim 9, further comprising delivering at least one of oxygen, vacuum suction, or saline to the selected area of scleral tissue from an oxygen source, a vacuum source, or a saline source, respectively, the oxygen source, the vacuum source, and the saline source being coupled to the at least one channel.

15. The method of claim 9, wherein the at least one insert includes a micro-fluidic material, wherein the at least one insert delivers the cross-linking agent to the selected area of scleral tissue according to micro-fluidic mechanisms.

16. The method of claim 9, wherein the illumination source provides at least one of near-infrared (NIR) light, visible (VIS) light, or ultraviolet (UV) light.

17. A system for conducting a corrective procedure for an eye, comprising:
a contact lens structure including at least one channel and at least one illumination fiber, the contact lens being configured for application over at least a cornea of the eye;
a cross-linking agent source coupled to the at least one channel; and
an illumination source coupled to the at least one illumination fiber,
wherein the contact lens structure delivers the cross-linking agent to a selected area of the eye via the at least one channel, and
the contact lens structure delivers photo-activating light from the illumination source to the selected area of the eye via the illumination fiber after the cross-linking agent has been delivered, the photo-activating light including one or more doses necessary for generating cross-linking activity in the selected are of the eye by activating the cross-linking agent and for activating TGF-β isoforms to improve health of extracellular matrices in the selected area of the eye.

18. The system of claim 17, further comprising an exogenous material source coupled to the at least one channel, wherein the contact lens structure delivers exogenous material to the selected area of the eye, the exogenous material generating exogenous cytokine augmented repair of extracellular matrices.

19. The system of claim 17, further comprising at least one of an oxygen source, a vacuum source, or a saline source coupled to the at least one channel, wherein the contact lens structure correspondingly delivers oxygen, vacuum suction, or saline to the selected area of the eye.

20. The system of claim 17, wherein the at least channel is a micro-fluidic channel that delivers the cross-linking agent to the selected area of the eye according to micro-fluidic mechanisms.
